(19) United States
(12) Patent Application Publication
(10) Pub. No.: US 2026/0295520 A1
(43) Pub. Date: Oct. 1, 2026

(54) ETHYLENE OXIDE ABATEMENT SYSTEMS

(71) Applicant: Boston Scientific Scimed, Inc., Maple Grove, MN (US)

(72) Inventors: Liming Crandall, Lino Lakes, MN (US); Joseph Thomas Delaney, JR., Minneapolis, MN (US); Rick William Dorn, Plymouth, MN (US); Vishal Bavishi, Natick, MA (US); Lauren Sfakis Lydecker, Upton, MA (US); Charlotte Innis, St. Paul, MN (US); Phillip Lawonn, Mounds View, MN (US)

(73) Assignee: Boston Scientific Scimed, Inc., Maple Grove, MN (US)

(21) Appl. No.: 19/632,823

(22) Filed: Mar. 30, 2026

Related U.S. Application Data
(60) Provisional application No. 63/780,856, filed on Mar. 31, 2025.

(51) Int. Cl. B01D 53/82; A61L 2/206; A61L 103/15; B01D 53/72; B01J 20/22; B01J 20/28; B01J 20/32; B65D 81/26

(52) U.S. Cl. CPC B01D 53/82; A61L 2/206; B01D 53/72; B01J 20/22; B01J 20/28033; B01J 20/3204; B01J 20/3219; B01J 20/3259; B65D 81/267; A61L 2103/15; A61L 2202/13; A61L 2202/181; B01D 2251/70; B01D 2257/708

(57) ABSTRACT

A system for abatement of sterilizing gas may include a medical device package having a medical device disposed therein and a sterilizing gas abatement member disposed adjacent to the medical device package. The sterilizing gas abatement member may comprise a base material having reactive silanol groups and a reactive acid end group covalently coupled to the reactive silanol groups. The sterilizing gas abatement member may be configured to abate a sterilizing gas present adjacent to the medical device.

### CROSS-REFERENCE TO RELATED APPLICATIONS

This application claims the benefit of priority under 35 U.S.C. § 119 of U.S. Provisional Application No. 63/780,856, filed Mar. 31, 2025, the entire disclosure of which is hereby incorporated by reference.

### TECHNICAL FIELD

The present disclosure pertains to medical devices, and methods for manufacturing and packaging medical devices. More particularly, the present disclosure pertains to ethylene oxide abatement systems.

### BACKGROUND

A wide variety of medical devices have been developed for medical use, for example, intravascular use. Some of these devices include guidewires, catheters, and the like. Most of these devices are required to be sterilized prior to use. Depending on the device type, material of construction, and device design, these devices are sterilized by any one of a variety of different sterilization methods, including, but not limited to, ethylene oxide (EtO) sterilization. EtO sterilization is a widely used sterilization method, among others, for medical device sterilization. There is an ongoing need to improve abatement technologies to reduce ethylene oxide exposure post-sterilization.

### BRIEF SUMMARY

This disclosure provides design, material, manufacturing, and packaging methods for ethylene oxide abatement. A sterilization abatement member may be positioned along with, near, or in proximity to the medical device primary package post-ethylene oxide sterilization. The sterilization abatement member may passively absorb residual off gassing ethylene oxide until the medical device is used and/or off-gassing ends. The end user can dispose the absorbent packet prior to using the medical device.

In an example, a system for abatement of sterilizing gas may include a medical device package having a medical device disposed therein and a sterilizing gas abatement member disposed adjacent to the medical device package, where the sterilizing gas abatement member may comprise a base material and a reactive acid end group, and where the sterilizing gas abatement member may be configured to abate a sterilizing gas present adjacent to the medical device.

Alternatively, or additionally to any of the examples above, in another example, the base material may comprise a porous substrate.

Alternatively, or additionally to any of the examples above, in another example, the base material may comprise a silica aerogel or fiberglass.

Alternatively, or additionally to any of the examples above, in another example, the reactive acid end group may be a sulfonic acid end group.

Alternatively, or additionally to any of the examples above, in another example, the reactive acid end group may be a phosphoric acid end group.

Alternatively, or additionally to any of the examples above, in another example, the medical device may comprise a sterilized medical device.

Alternatively, or additionally to any of the examples above, in another example, the sterilizing gas may comprise ethylene oxide.

Alternatively, or additionally to any of the examples above, in another example, the medical device package may be disposed within the sterilizing gas abatement member.

Alternatively, or additionally to any of the examples above, in another example, the sterilizing gas abatement member may be disposed about the medical device package.

Alternatively, or additionally to any of the examples above, in another example, the sterilizing gas abatement member may include a blanket.

In an example, a method for forming a sterilizing gas abatement member may include reacting a base material having reactive silanol groups on a surface thereof with a reactive silicon compound, where the reactive silicon compound may include a mineral acid derivative, and where the silicon compound may covalently couple to the reactive silanol groups.

Alternatively, or additionally to any of the examples above, in another example, the reactive silicon compound may comprise at least one of: trimethoxysilyl, triethoxysilyl, bromodimethylsilyl, tribromosilyl, chlorodimethylsilyl, or trichlorosilyl.

Alternatively, or additionally to any of the examples above, in another example, the mineral acid derivative may comprise at least one of: sulfonic acid, sulfonyl chloride, sulfonyl esters, phosphoryl chloride, or phosphate esters.

Alternatively, or additionally to any of the examples above, in another example, the base material may comprise a silica aerogel.

Alternatively, or additionally to any of the examples above, in another example, the base material may comprise fiberglass.

In an example, a system for abatement of sterilizing gas may include a medical device package having a medical device disposed therein and a sterilizing gas abatement member disposed adjacent to the medical device package, where the sterilizing gas abatement member may comprise a base material and a reactive acid end group, and where the sterilizing gas abatement member may be configured to abate a sterilizing gas present adjacent to the medical device.

Alternatively, or additionally to any of the examples above, in another example, the base material may comprise a porous substrate.

Alternatively, or additionally to any of the examples above, in another example, the base material may comprise a silica aerogel or fiberglass.

Alternatively, or additionally to any of the examples above, in another example, the reactive acid end group may be a sulfonic acid end group.

Alternatively, or additionally to any of the examples above, in another example, the reactive acid end group may be a phosphoric acid end group.

Alternatively, or additionally to any of the examples above, in another example, the medical device may comprise a sterilized medical device.

Alternatively, or additionally to any of the examples above, in another example, the sterilizing gas may comprise ethylene oxide.

Alternatively, or additionally to any of the examples above, in another example, the medical device package may be disposed within the sterilizing gas abatement member.

Alternatively, or additionally to any of the examples above, in another example, the sterilizing gas abatement member may be disposed about the medical device package.

Alternatively, or additionally to any of the examples above, in another example, the sterilizing gas abatement member may include a blanket.

In an example, a method for forming a sterilizing gas abatement member may include reacting a base material having reactive silanol groups on a surface thereof with a reactive silicon compound, where the reactive silicon compound may include a mineral acid derivative, and where the silicon compound may covalently couple to the reactive silanol groups.

Alternatively, or additionally to any of the examples above, in another example, the reactive silicon compound may comprise at least one of: trimethoxysilyl, triethoxysilyl, bromodimethylsilyl, tribromosilyl, chlorodimethylsilyl, or trichlorosilyl.

Alternatively, or additionally to any of the examples above, in another example, the mineral acid derivative may comprise at least one of: sulfonic acid, sulfonyl chloride, sulfonyl esters, phosphoryl chloride, or phosphate esters.

Alternatively, or additionally to any of the examples above, in another example, the base material may comprise a silica aerogel.

Alternatively, or additionally to any of the examples above, in another example, the base material may comprise fiberglass.

In an example, a system for abatement of sterilizing gas may include a medical device package having a medical device disposed therein and a sterilizing gas abatement member disposed adjacent to the medical device, where the sterilizing gas abatement member may comprise a porous base material having reactive silanol groups and a reactive acid end group, and where the sterilizing gas abatement member may be configured to abate a sterilizing gas present adjacent to the medical device.

Alternatively, or additionally to any of the examples above, in another example, the reactive acid end groups may comprise sulfonic acid end groups or phosphoric acid end groups.

Alternatively, or additionally to any of the examples above, in another example, the base material may have a density in the range of about 0.001 to about 0.5 g/cm³.

Alternatively, or additionally to any of the examples above, in another example, the base material may comprise a silica aerogel.

Alternatively, or additionally to any of the examples above, in another example, the base material may comprise fiberglass.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 schematically depicts an example medical device disposed within a medical device package post-sterilization;

FIG. 2 is a chemical equation illustrating the reaction between reactive silanol groups and an illustrative reactive agent to form an acid functionalizing agent;

FIG. 3 is a chemical equation illustrating the reaction between reactive silanol groups and another illustrative reactive agent to form an acid functionalizing agent;

FIG. 4 is a chemical equation illustrating the reaction between reactive silanol groups and another illustrative reactive agent to form an acid functionalizing agent;

FIG. 5 schematically depicts a plurality of medical device packages with a sterilizing gas abatement member disposed adjacent the medical device packages;

FIG. 6 schematically depicts a plurality of medical device packages with a sterilizing gas abatement member disposed adjacent the medical device packages; and

FIG. 7 schematically depicts a plurality of medical device packages with a sterilizing gas abatement member disposed adjacent the medical device packages.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Medical devices such as guidewires, catheters, balloon catheters, stents, stent delivery systems, and the like are sterilized prior to use to eliminate the presence of microorganisms and to provide efficacious and safe use. A number of different sterilization techniques may be used. One example sterilization technique is the use of ethylene oxide (EtO) gas. Ethylene oxide may be highly effective for most materials that are sensitive to humidity and temperature. Ethylene oxide is also strictly regulated by the Occupational Safety and Health Administration (OSHA) and Environmental Protection Agency (EPA) due to its hazardous properties, including, but not limited to, being flammable and carcinogenic. While effective for sterilization, residual ethylene oxide gas can be present (e.g., off-gas) from the devices and/or packaging after the sterilization procedure. In general, the relative amount of residual ethylene oxide gas present may be considered low. However, in a manufacturing setting where relatively large quantities of sterilized and packaged medical devices may be present, the levels of residual ethylene oxide gas can rise to undesirable levels.

One of the common abatement technologies for ethylene oxide is chemical gas scrubbers, using sulfuric acid as a catalyst which stabilizes and breaks down ethylene oxide into ethylene glycol and ethylene glycol derivatives. The EPA requires additional personal protective equipment (PPE), such as a self-contained breathing apparatus or a supplied airline respirator, to mitigate potential inhalation exposure risks to workers involved in the ethylene oxide sterilization process. In addition, the EPA also requires real-time monitoring of ethylene oxide with a limit of quantification at or below 10 parts per billion (ppb) in the workplace, otherwise workers are required to wear PPE. For worker safety and to comply with EPA regulations, the sterilized goods are often stored and allowed to off-gas (emit residual ethylene oxide). Disclosed herein are systems for abatement of residual sterilizing gas, for example, ethylene oxide.

Medical devices may be packaged in a number of different ways. In some cases, medical devices may be packaged in a primary package prior to sterilization. Primary packaging may include, but is not limited to, pouches, blister packs, bags, or the like. After sterilization, the primary packages may be grouped and placed into secondary packaging. Secondary packaging may include, but is not limited to, cardboard boxes, plastic trays, or the like. Each secondary packaging container may include one or more medical devices each disposed within a primary package. Further, the secondary packaging containers may be grouped and placed into tertiary packaging. Tertiary packaging containers may include, but are not limited to, pallets, stretch wrap, or other containers. Each tertiary packaging container may include two or more secondary packaging containers.

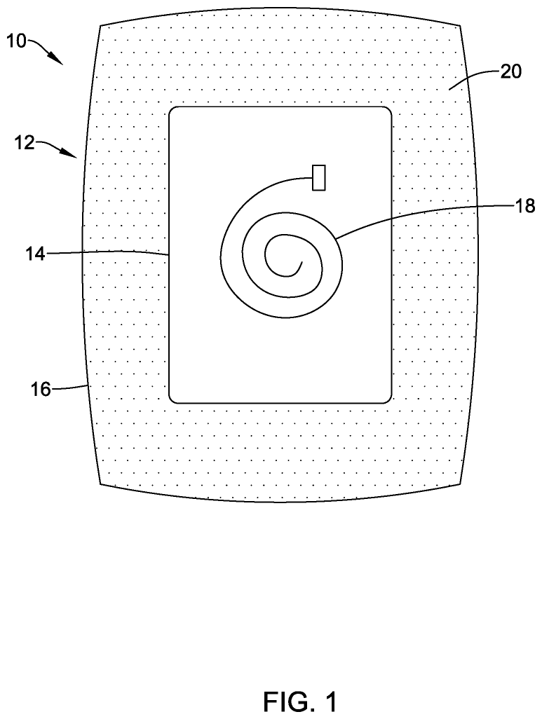

FIG. 1 schematically depicts an example system 10. The system 10 may include a medical device package 12. In this example, the medical device package 12 may include an inner packaging member or pouch 14 and an outer packaging member or pouch 16. Examples are contemplated that utilize more or fewer components. For example, the outer packaging member 16 may be omitted. A medical device 18, depicted schematically, may be disposed within the medical device package 12. The medical device 18 may be any of a variety of different medical devices such as guidewires, catheters, balloon catheters, stents, stent delivery systems, and the like. These are just examples. Other forms of medical devices are contemplated. In this example, the medical device 18 is shown disposed within the inner packaging member/pouch 14. In some examples, the outer packaging member/pouch 16 may be formed from a sterilizing gas abatement substance or member 20. In such an example, the inner packaging member/pouch 14 may be placed in the outer packaging member/pouch 16 after the sterilization procedure. However, the outer packaging member/pouch 16 need not be formed from a sterilizing gas abatement substance or member 20.

The sterilizing gas abatement member 20 may have a variety of forms. In general, the sterilizing gas abatement member 20 may be configured to react with ethylene oxide gas and neutralize the ethylene oxide gas. In some examples, the sterilizing gas abatement member 20 may react with the ethylene oxide gas to form ethylene glycol and such derivates. However, other chemical reactions may occur. In some instances, the sterilizing gas abatement member 20 may include an aerogel, fiberglass, or other material that has been modified to include an acid functionalizing agent.

Generally, the sterilizing gas abatement substance 20 may be a functionalized base material. The base material may include a porous, lightweight substrate having reactive thermostable silanol groups on surface thereof. The silanol groups may be covalently coupled via an SN2 coupling reaction with reactive silicon compounds to form protonated acid end groups. The functionalized material (e.g., including the protonated acid end groups) may break down ethylene oxide into less harmful substances.

Aerogels are a class of porous, solid materials having a low density. Some aerogels may be silica aerogels. Generally, silica aerogels may be formed from silica particles that are connected together. In some cases, the silica particles may be extremely small, ranging from about 2 to about 50 nanometers in diameter. The silica nanoparticles may cluster together into large spherical particles having a diameter in the range of about 50 nanometers to about 2 micrometers. The silica nanoparticles connect in a manner which creates a highly porous network. Low density silica aerogels may have a density in the range of about 0.001 to about 0.01 grams per cubic centimeter (g/cm³). Medium density silica aerogels may have a density in the range of about 0.01 to about 0.1 g/cm³. High density silica aerogels may have a density in the range of about 0.1 to about 0.5 g/cm³ or more than 0.5 g/cm³. The surface of the silica particles may be covered with thermostable reactive silanol (Si—OH) groups. The silanol groups of the aerogel may be reacted with another material, referred to herein as a reactant or reactive agent, to form a material having an acid functionalizing agent that can react with ethylene oxide gas to neutralize or abate the ethylene oxide gas.

Fiberglass is a composite material including a plurality of very fine glass fibers and a resin. Fiberglass may include thermostable reactive silanol groups on the surface thereof that can be reacted with another material, referred to herein as a reactant or reactive agent, to form a material having an acid functionalizing agent that can react with ethylene oxide gas to neutralize or abate the ethylene oxide gas. Further, fiberglass is available in a wide range of formats including varying flexibilities. In some cases, fiberglass may have a density in the range of about 2.5 to about 2.76 g/cm³.

It is contemplated that to create a suitable acid functionalizing agent to react with ethylene oxide, the reactant or reactive agent may include a reactive silicon with a suitable leaving group for an SN2 reaction and a mineral acid derivative that can be protonated after coupling. In one illustrative example, the reactive silanol groups of a silica aerogel, fiberglass, or other reactive silanol containing material, can be reacted with 2-(4-chlorosulfonylphenyl)ethyltrimethoxysilane (CSPTMS) and aqueous acid to attach a 4-(2-silyethyl)benzenesulfonic acid group to the silica surface. CSPTMS has the following chemical structure:

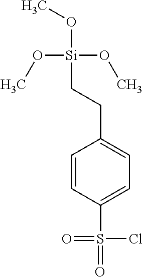 [SMILES: CO[Si](CCc1ccc(S(=O)(=O)Cl)cc1)(OC)OC]

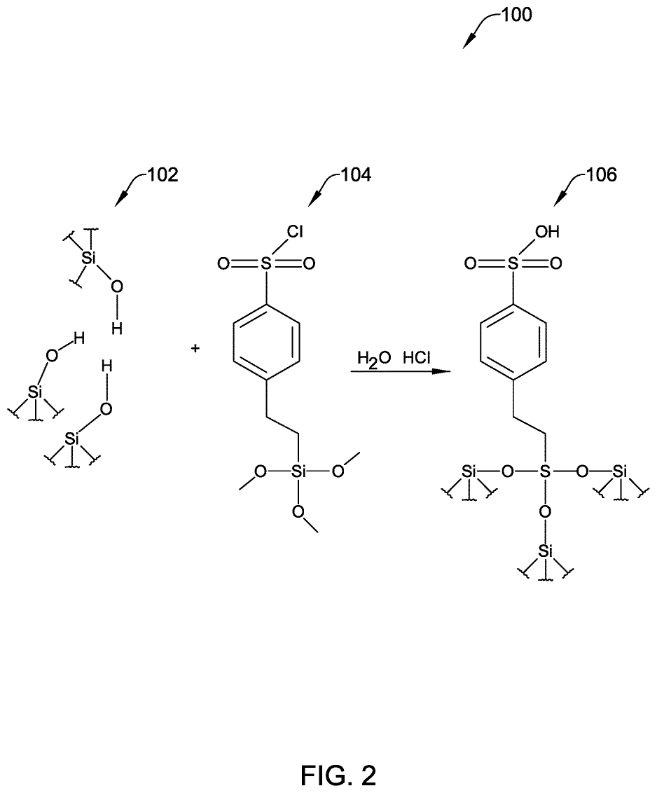

The example reaction 100 between the silanol groups 102 of the aerogel or fiberglass and the reactant or reactive agent 104, CSPTMS, is shown FIG. 2. The aryl sulfonyl chloride of the reactive agent 104 is replaced with an aryl sulfonic acid group to form a product 106 (e.g., a sterilizing gas abatement member 20) having a sulfonic acid catalyst site. The reactive silicon of the reactive agent reacts with the silanol groups of the base material (e.g., silica aerogel, fiberglass, or the like). The product 106 (e.g., a sterilizing gas abatement member 20) may include an organosulfonic group covalently bonded to the surface of the silica aerogel. Water and hydrochloric acid may be used as additional reagents within the reaction.

In an example reaction, 3 grams of 50 weight percent CSPTMS in dichloromethane was mixed with 5 grams of a silica-based aerogel and 3 milliliters of dichloromethane followed by 3 milliliters of 2 molar hydrochloric acid. The system was allowed to react at 40° for 1.5 hours and then overnight at room temperature prior to rinsing with water and drying at room temperature.

In another illustrative example, the reactive silanol groups of a silica aerogel, fiberglass, or other reactive silanol containing material, can be reacted with (4-diethoxyphosphorylphenyl)-triethoxy silane and aqueous acid to attach to to form an aryl phosphoric acid group attached to the silica surface. (4-diethoxyphosphorylphenyl)-triethoxy silane has the following chemical structure:

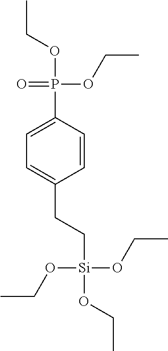 [SMILES: CCO[Si](CCc1ccc(P(=O)(OCC)OCC)cc1)(OCC)OCC]

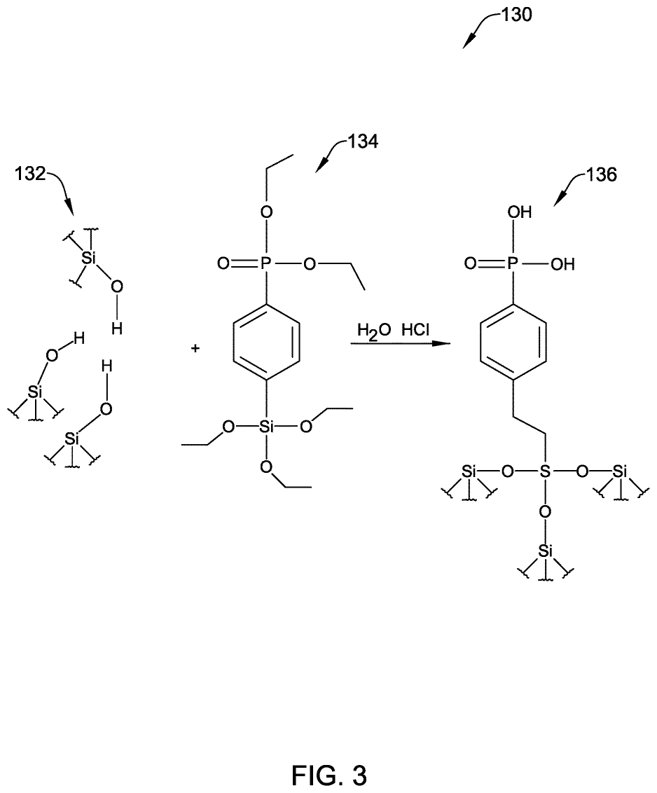

The example reaction 130 between the silanol groups 132 of the base material (e.g., silica aerogel, fiberglass, or the like) and the reactant or reactive agent 134, (4-diethoxyphosphorylphenyl)-triethoxy silane, is shown FIG. 3. The aryl phosphate ethoxy groups are replaced with an aryl phosphoric acid group to form a product 136 (e.g., a sterilizing gas abatement member 20) having a phosphoric acid catalyst site. The product 136 (e.g., a sterilizing gas abatement member 20) may include an organophosphoric group covalently bonded to the surface of the silica aerogel. Water and hydrochloric acid may be used as additional reagents within the reaction.

In another illustrative example, the reactive silanol groups of a silica aerogel, fiberglass, or other silanol containing material, can be reacted with a mineral acid-silanol lactone with ring opening forming a sulfonic acid catalyst site. In some cases, the mineral acid-silanol lactone may be 1,3-benzoxathiasilole, 3,3-dimethyl-, 1,1-dioxide having the following chemical structure:

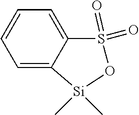

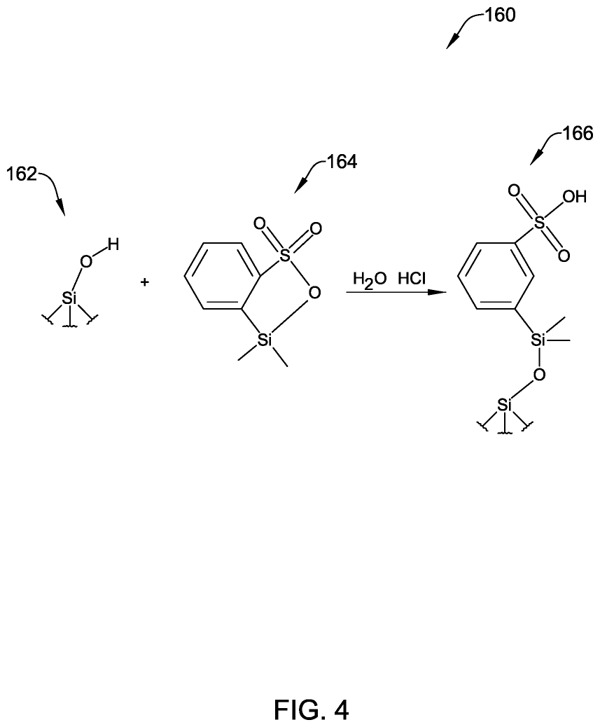

The example reaction 160 between the silanol groups 162 of the base material (e.g., silica aerogel, fiberglass, or the like) and the reactant or reactive agent 164, 1,3-benzoxathiasilole, 3,3-dimethyl-, 1,1-dioxide, is shown FIG. 4. In this example, a heterocyclic portion of the compound opens at the oxygen molecule. During the reaction, a hydroxyl group bonds with the sulfur to form a product 166 (e.g., a sterilizing gas abatement member 20) having a sulfonic acid catalyst site. The product 166 (e.g., a sterilizing gas abatement member 20) may include a sulfonic acid group covalently bonded to the surface of the silica aerogel. Water and hydrochloric acid may be used as additional reagents within the reaction.

Other reactants having reactive silicon groups and mineral acid derivatives may also be used. Reactive silicon groups may include, but are not limited to, ortho esters of silanol (e.g., trimethoxysilyl, or triethoxysilyl), bromodimethylsilyl, tribromosilyl, chlorodimethylsilyl, trichlorosilyl, or the like. Mineral acid derivatives may include, but are not limited to, sulfur or phosphorus-based moieties such as sulfonic acid, sulfonyl chloride, sulfonyl esters (including methyl esters and trimethylsilyl esters), phosphoryl chloride, phosphate esters, or the like. In some cases, phosphoric acid may be directly embedded in the silica support. Another illustrative reactant may be 2-(4-chlorosulfonylphenyl)ethyltrichlorosilane, having a chemical abstracts service (CAS) registry number of 79793-00-3 and the following chemical structure:

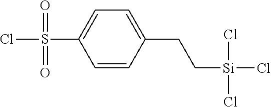 [SMILES: O=S(=O)(Cl)c1ccc(CC[Si](Cl)(Cl)Cl)cc1]

Another illustrative reactant may be 4-(trichlorosilyl)benzenesulfonic acid (CAS #220877-47-4) having the following chemical structure:

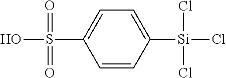 [SMILES: O=S(=O)(O)c1ccc([Si](Cl)(Cl)Cl)cc1]

Another illustrative reactant may be 4-(trimethoxysilyl)benzenesulfonyl chloride (CAS #1132650-31-7) having the following chemical structure:

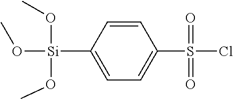 [SMILES: CO[Si](OC)(OC)c1ccc(S(=O)(=O)Cl)cc1]

Another illustrative reactant may be 4-(2-(triethoxysilyl)ethyl)benzene-1-sulfonyl chloride (CAS #597539-49-6) having the following chemical structure:

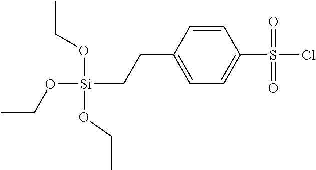 [SMILES: CCO[Si](CCc1ccc(S(=O)(=O)Cl)cc1)(OCC)OCC]

Another illustrative reactant may be ethyl 4-[2-(trichlorosilyl)ethyl]benzenesulfonate (CAS #1265625-60-2) having the following chemical structure:

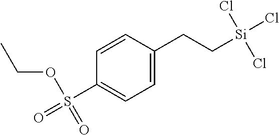 [SMILES: CCOS(=O)(=O)c1ccc(CC[Si](Cl)(Cl)Cl)cc1]

Another illustrative reactant may be 4-[2-(dichloromethylsilyl)ethyl]benzenesulfonyl chloride (CAS #220289-46-3) having the following chemical structure:

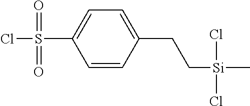 [SMILES: C[Si](Cl)(Cl)CCc1ccc(S(=O)(=O)Cl)cc1]

Another illustrative reactant may be 3-(trihydroxysilyl)-1-propanesulfonic acid (CAS #70942-24-4) having the following chemical structure:

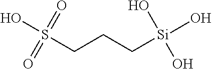 [SMILES: O=S(=O)(O)CCC[Si](O)(O)O]

Another illustrative reactant may be 4-[2-(trimethoxysilyl)ethyl]benzenesulfonic acid (CAS #58556-70-0) having the following chemical structure:

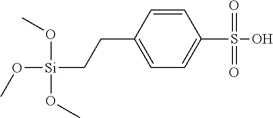 [SMILES: CO[Si](CCc1ccc(S(=O)(=O)O)cc1)(OC)OC]

Another illustrative reactant may be 1-methylethyl-3-(trimethoxysilyl)-1-propanesulfonate (CAS #189344-76-1) having the following chemical structure:

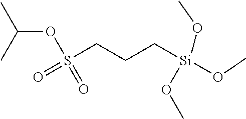 [SMILES: CO[Si](CCCS(=O)(=O)OC(C)C)(OC)OC]

Another illustrative reactant may be 3H-2,1,3-benzoxathiasilole-3,3,-dimethyl-1,1-dioxide (CAS #55562-97-5) having the following chemical structure:

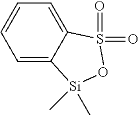

Another illustrative reactant may be 3,3,5,5-tetramethyl-[1,2,5]oxathiasilolo[4,3-f][2,1,3]benzoxathiasilole 1,1,7,7-tetraoxide (CAS #55563-02-5) having the following chemical structure:

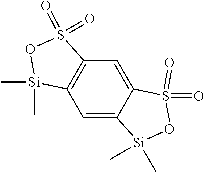 [SMILES: C[Si]1(C)OS(=O)(=O)c2cc3c(cc21)[Si](C)(C)OS3(=O)=O]

Another illustrative reactant may be (4-diethoxyphosphorylphenyl)-hydroxy-dimethyl-silane (CAS #914656-94-3) having the following chemical structure:

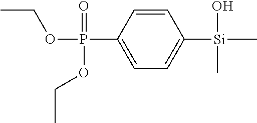 [SMILES: CCOP(=O)(OCC)c1ccc([Si](C)(C)O)cc1]

Another illustrative reactant may be diethyl P-[4-[2-(trimethoxysilyl)ethyl]phenyl]phosphonate (CAS #1265625-62-4) having the following chemical structure:

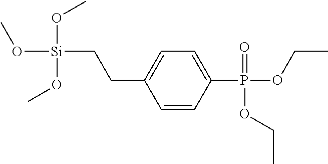 [SMILES: CCOP(=O)(OCC)c1ccc(CC[Si](OC)(OC)OC)cc1]

Another illustrative reactant may be diethyl P-[4-[2-(triethoxysilyl)ethyl]phenyl]phosphonate (CAS #1265625-61-3) having the following chemical structure:

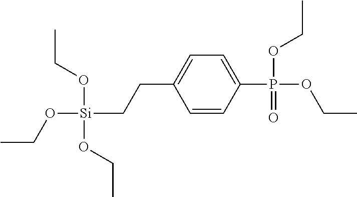 [SMILES: CCO[Si](CCc1ccc(P(=O)(OCC)OCC)cc1)(OCC)OCC]

Another illustrative reactant may be P-[3-(trimethoxysilyl)phenyl]phosphonic acid (CAS #1631161-85-7) having the following chemical structure:

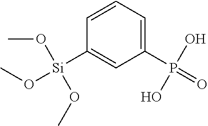 [SMILES: CO[Si](OC)(OC)c1cccc(P(=O)(O)O)c1]

The above reactants are just some examples of illustrative reactive agents that can be reacted with a silica aerogel, fiberglass, or other materials having reactive silanol groups to form a sterilizing gas abatement substance or member 20 having an acid functionalizing agent for oxide surfaces. The sterilizing gas abatement substance 20 may be positioned in close proximity to the sterilized primary package 14 to scavenge ethylene oxide gas as it diffuses from the primary package 14. However, this is not required. In the illustrated embodiments of FIG. 1, the sterilizing gas abatement substance 20 may surround the primary package 14 or the primary package 14 may be disposed within the sterilizing gas abatement substance 20. Starting with a porous material or open pore structure, such as a silica aerogel or fiberglass, may increase the surface area or provide a large surface area for the ethylene gas to diffuse into and/or through. Further, increasing the surface area may increase the number of reactive silanol groups available to react with the reactive component and thus increase the acid functionalizing agents available to react with and neutralize the ethylene oxide gas. Additionally, starting with a porous material may allow for a minimal pressure difference across the sterilizing gas abatement substance 20 to allow for diffusion of the ethylene oxide gas into the sterilizing gas abatement substance 20. Said differently, the porous nature of silica aerogels and fiberglass may provide a high surface area containing reactive thermostable silanol groups that can be functionalized with acid groups. This high surface area may maximize the potential contact between the functionalized acid groups and the ethylene oxide gas. This may make the abatement process more efficient. However, it is not required that the base material be porous. Any substance having reactive silanol groups may be used as the base material in place of or in addition to a porous base material.

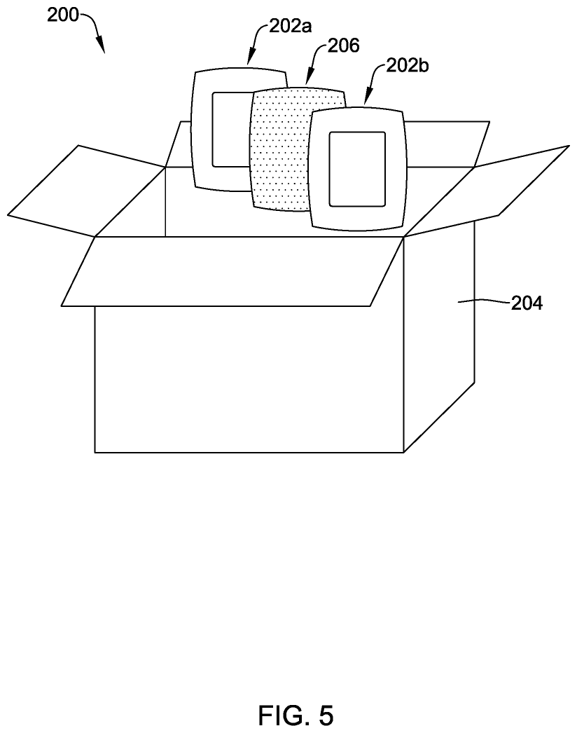

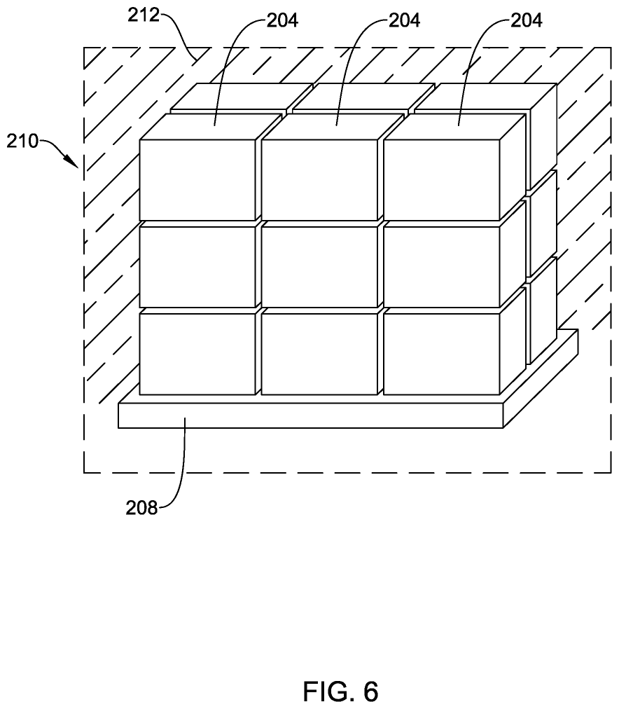

FIG. 5 illustrates a system 200 including a plurality of medical device packages 202a, 202b. The medical device packages 202a, 202b may be similar in form and function to the medical device package 12 described herein. If so provided, an outer package/pouch may or may not be formed from a sterilizing gas abatement member 20, as desired. The medical device packages 202a, 202b may be disposed within a container 204 such as box. The container 204 may include one or more medical device packages 202a, 202b. In some instances, multiple containers 204 may be stacked together onto a pallet 208 as shown in FIG. 6. It can be appreciated that assemblies such as a plurality of medical device packages (e.g., medical device packages 202a, 202b), a plurality of medical device packages within a box/container 204, a plurality of boxes/containers 204 disposed on a pallet 208 could have the ability to release a relatively large amount of ethylene oxide gas after sterilization. Disposing a sterilizing gas abatement member 206 adjacent to the medical device packages 202a, 202b may significantly reduce the level of residual ethylene oxide gas present at or near such assemblies. In some cases, the sterilizing gas abatement member 206 may be provided as a separate component from the medical device packages 202a, 202b. For example, one or more sterilizing gas abatement members 206 may be provided as one or more sheets, blankets, foams, liners, wraps, pellets, beads, sponges, packing peanuts, sachets, combination thereof, or other structure within the container 204 as a separate component from the medical device packages 202a, 202b. It is contemplated that the sterilizing gas abatement member 206 may be provided in addition to or in place of a sterilizing gas abatement member 20 surrounding the primary packaging.

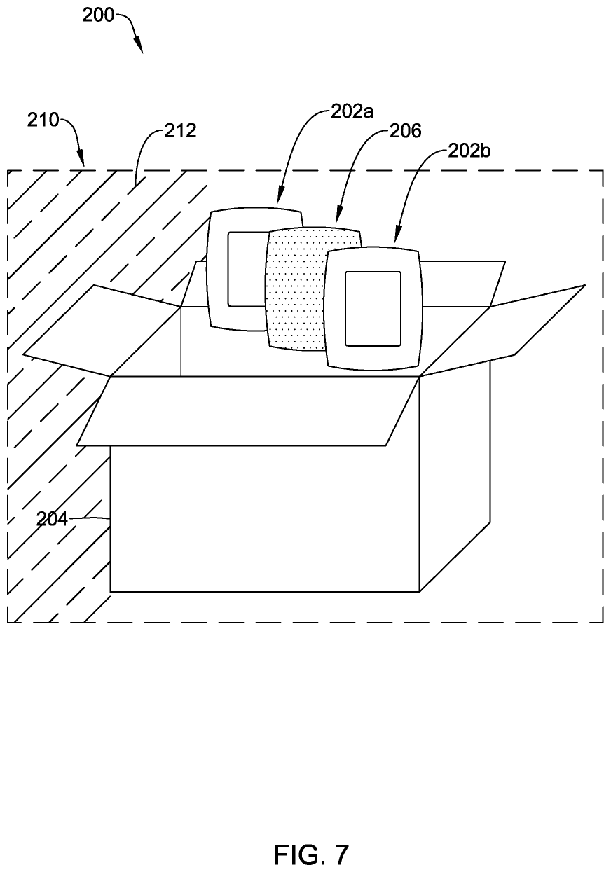

While the sterilizing gas abatement member 20 surrounding the primary packaging and/or the sterilizing gas abatement member 206 disposed within the container 204 may be sufficient to abate residual ethylene oxide gas, other abatement structures are contemplated. For example, FIG. 7 illustrates the system 200 of FIG. 5 including a plurality of medical device packages 202a, 202b disposed within the container 204 along with a sterilizing gas abatement member 206. In this example, a sterilizing gas abatement member 210 may be disposed along, near, or in proximity to the container 204. The sterilizing gas abatement member 210 may take the form of a sterilizing gas abatement blanket 210 having or being formed from a sterilizing gas abatement substance 212. Other form factors are contemplated for the sterilizing gas abatement member 210 includes wraps, boxes, containers, etc. with the sterilizing gas abatement substance 212 incorporated therein. The sterilizing gas abatement member 210 may be provided in place of one or both of the sterilizing gas abatement member 20 and the sterilizing gas abatement member 206 or in addition to one or both of the sterilizing gas abatement member 20 and the sterilizing gas abatement member 206. In some cases, the sterilizing gas abatement member 210 may be placed in the head space of, for example, trucks, shipping container, or the like, to abate ethylene oxide during transit. Said differently, the sterilizing gas abatement member 210 need not surround the primary packaging or containers 204.

In some instances, the sterilizing gas abatement substance 212 may comprise a porous material that can be incorporated into or entirely form the sterilizing gas abatement blanket 210. With the sterilizing gas abatement substance 212 woven/incorporated into or otherwise forming the sterilizing gas abatement blanket 210, the sterilizing gas abatement blanket 210 can be disposed on, about, form-fitted to, positioned near, etc. various assemblies of medical device packages. For example, the sterilizing gas abatement blanket 210 can be disposed about, around, surround, or the like, a singular medical device package 202a, 202b. In some of these and in other instances, the sterilizing gas abatement blanket 210 can be disposed about, around, surround, or the like, a box or container 204 having a plurality of medical device packages 202a, 202b disposed therein (e.g., as shown in FIG. 7). In some instances, multiple containers 204 may be stacked together onto a pallet 208, as shown in FIG. 6. In such instances, the sterilizing gas abatement blanket 210 may be disposed along, onto, near, or over the pallet 208 and containers 204 to abate ethylene oxide gas. In some cases, the sterilizing gas abatement member 210 may be placed in the head space of, for example, trucks, shipping container, or the like, to abate ethylene oxide during transit.

The sterilizing gas abatement blanket 210 can suitably scavenge ethylene oxide gas that may diffuse from the medical device packages (e.g., medical device packages 202a, 202b). The sterilizing gas abatement blanket 210 may have a suitable surface area to cover the medical device packages (e.g., medical device packages 202a, 202b), boxes or containers 204 (e.g., having a plurality of medical device packages 202a, 202b disposed therein), multiple boxes or containers 204 stacked together onto a pallet 208, etc. In addition, the porous nature of the sterilizing gas abatement blanket 210 (e.g., when such a form factor is utilized) may expand/increase the surface area onto which ethylene oxide can interact with the fabric and, in particular, the sterilizing gas abatement substance 212.

Each of the sterilizing gas abatement members 20, 206 or sterilizing gas abatement blanket 210 may be formed from the same material or different materials. Each of the sterilizing gas abatement members 20, 206 or sterilizing gas abatement blanket 210 may include a porous base material (e.g., silica aerogel, fiberglass, or other material having reactive silanol groups) that has been an acid functionalizing agent that can react with ethylene oxide gas as described herein. Further, the sterilizing gas abatement members 20, 206 or sterilizing gas abatement blanket 210 may be disposed of prior to use of the packaged medical device.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for abatement of sterilizing gas, the system comprising:
a medical device package having a medical device disposed therein; and
a sterilizing gas abatement member disposed adjacent to the medical device package, the sterilizing gas abatement member comprising a base material and a reactive acid end group;
wherein the sterilizing gas abatement member is configured to abate a sterilizing gas present adjacent to the medical device.

2. The system of claim 1, wherein the base material comprises a porous substrate.

3. The system of claim 2, wherein the base material comprises a silica aerogel or fiberglass.

4. The system of claim 1, wherein the reactive acid end group is a sulfonic acid end group.

5. The system of claim 1, wherein the reactive acid end group is a phosphoric acid end group.

6. The system of claim 1, wherein the medical device comprises a sterilized medical device.

7. The system of claim 1, wherein the sterilizing gas comprises ethylene oxide.

8. The system of claim 1, wherein the medical device package is disposed within the sterilizing gas abatement member.

9. The system of claim 1, wherein the sterilizing gas abatement member is disposed about the medical device package.

10. The system of claim 1, wherein the sterilizing gas abatement member includes a blanket.

11. A method for forming a sterilizing gas abatement member, the method comprising:
reacting a base material having reactive silanol groups on a surface thereof with a reactive silicon compound, the reactive silicon compound including a mineral acid derivative;
wherein the silicon compound covalently couples to the reactive silanol groups.

12. The method of claim 11, wherein the reactive silicon compound comprises at least one of: trimethoxysilyl, triethoxysilyl, bromodimethylsilyl, tribromosilyl, chlorodimethylsilyl, or trichlorosilyl.

13. The method of claim 11, wherein the mineral acid derivative comprises at least one of: sulfonic acid, sulfonyl chloride, sulfonyl esters, phosphoryl chloride, or phosphate esters.

14. The method of claim 11, wherein the base material comprises a silica aerogel.

15. The method of claim 11, wherein the base material comprises fiberglass.

16. A system for abatement of sterilizing gas, the system comprising:
a medical device package having a medical device disposed therein; and
a sterilizing gas abatement member disposed adjacent to the medical device, the sterilizing gas abatement member comprising a porous base material having reactive silanol groups and a reactive acid end group;
wherein the sterilizing gas abatement member is configured to abate a sterilizing gas present adjacent to the medical device.

17. The system of claim 16, wherein the reactive acid end groups comprise sulfonic acid end groups or phosphoric acid end groups.

18. The system of claim 16, wherein the base material has a density in the range of about 0.001 to about 0.5 g/cm³.

19. The system of claim 16, wherein the base material comprises a silica aerogel.

20. The system of claim 16, wherein the base material comprises fiberglass.
